SEQUENCE LISTING (32 sequences)

SEQ ID NO: 1 - DNA, 1408 bp, Hominidae
  CDS (1)..(900)
aaacagaaggggaggtgcagtttcagaacccagccagcctctctcttgctgcctagcctcctgccggcctcatcttcgcccagccaaccccgcctggagccctatggccaactgcgagttcagcccggtgtccggggacaaaccctgctgccggctctctaggagagcccaactctgtcttggcgtcagtatcctggtcctgatcctcgtcgtggtgctcgcggtggtcgtcccgaggtggcgccagcagtggagcggtccgggcaccaccaagcgctttcccgagaccgtcctggcgcgatgcgtcaagtacactgaaattcatcctgagatgagacatgtagactgccaaagtgtatgggatgctttcaagggtgcatttatttcaaaacatccttgcaacattactgaagaagactatcagccactaatgaagttgggaactcagaccgtaccttgcaacaagattcttctttggagcagaataaaagatctggcccatcagttcacacaggtccagcgggacatgttcaccctggaggacacgctgctaggctaccttgctgatgacctcacatggtgtggtgaattcaacacttccaaaataaactatcaatcttgcccagactggagaaaggactgcagcaacaaccctgtttcagtattctggaaaacggtttcccgcaggtttgcagaagctgcctgtgatgtggtccatgtgatgctcaatggatcccgcagtaaaatctttgacaaaaacagcacttttgggagtgtggaagtccataatttgcaaccagagaaggttcagacactagaggcctgggtgatacatggtggaagagaagattccagagacttatgccaggatcccaccataaaagagctggaatcgattataagcaaaaggaatattcaattttcctgcaagaatatctacagacctgacaagtttcttcagtgtgtgaaaaatcctgaggattcatcttgcacatctgagatctgagccagtcgctgtggttgttttagctccttgactccttgtggtttatgtcatcatacatgactcagcatacctgctggtgcagagctgaagattttggagggtcctccacaataaggtcaatgccagagacggaagcctttttccccaaagtcttaaaataacttatatcatcagcatacctttattgtgatctatcaatagtcaagaaaaattattgtataagattagaatgaaaattgtatgttaagttacttcactttaattctcatgtgatccttttatgttatttatatattggtaacatcctttctattgaaaaatcaccacaccaaacctctcttattagaacaggcaagtgaagaaaagtgaatgctcaagtttttcagaaagcattacatttcc

SEQ ID NO: 2 - protein, 300 aa, Hominidae
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 3 - DNA, 61 bp, Hominidae
cgcccgccgcgccccgcgcccgtcccgccgcccccgcccgatcttcgcccagccaaccccg

SEQ ID NO: 4 - DNA, 21 bp, Hominidae
accggtgcgccttagtcgcca

SEQ ID NO: 5 - DNA, 21 bp, Hominidae
tagactgcatgttagacgaga

SEQ ID NO: 6 - DNA, 62 bp, Hominidae
cgcccgccgcgccccgcgcccgtcccgccgcccccgcccgtttggacctatgaattgttacc

SEQ ID NO: 7 - DNA, 21 bp, Hominidae
gacatgctaaattgatctcag

SEQ ID NO: 8 - DNA, 60 bp, Hominidae
cgcccgccgcgccccgcgcccgtcccgccgcccccgcccgcagcagaagtcactctgttc

SEQ ID NO: 9 - DNA, 21 bp, Hominidae
ccattctccagcctccgtctt

SEQ ID NO: 10 - DNA, 62 bp, Hominidae
cgcccgccgcgccccgcgcccgtcccgccgcccccgcccgcaagcactgactgagtaacgtc

SEQ ID NO: 11 - DNA, 22 bp, Hominidae
aaactgctggaggatggtgatt

SEQ ID NO: 12 - DNA, 63 bp, Hominidae
cgcccgccgcgccccgcgcccgtcccgccgcccccgcccgttcactgtgatatttgcaacagg

SEQ ID NO: 13 - DNA, 23 bp, Hominidae
ggttgatgtttggggttctttgt

SEQ ID NO: 14 - DNA, 64 bp, Hominidae
cgcccgccgcgccccgcgcccgtcccgccgcccccgcccgtgtggattcttttgtggactgatt

SEQ ID NO: 15 - DNA, 61 bp, Hominidae
cgcccgccgcgccccgcgcccgtcccgccgcccccgcccgttgtccagggcgtgctacaaa

SEQ ID NO: 16 - DNA, 21 bp, Hominidae
agattcacacagccctccaag

SEQ ID NO: 17 - DNA, 62 bp, Hominidae
cgcccgccgcgccccgcgcccgtcccgccgcccccgcccgttagcgaattggacgacagatg

SEQ ID NO: 18 - DNA, 22 bp, Hominidae
tctggcattgaccttattgtgg

SEQ ID NO: 19 - DNA, 22 bp, Hominidae
ctccgccactctcctgcacaca

SEQ ID NO: 20 - DNA, 20 bp, Hominidae
gggcctccagcagaagtcac

SEQ ID NO: 21 - DNA, 21 bp, Hominidae
ttgtccagggcgtgctacaaa

SEQ ID NO: 22 - DNA, 66 bp, Hominidae
ttagcgaattggacgacagatgtatcctacggtctcttgatttccttttttgctttcttgtcatag

SEQ ID NO: 23 - DNA, 15 bp, Homo sapiens
actcagaccgtacct

SEQ ID NO: 24 - DNA, 15 bp, Homo sapiens
actcagactgtacct

SEQ ID NO: 25 - DNA, 15 bp, Homo sapiens
gtccagcgggacatg

SEQ ID NO: 26 - DNA, 15 bp, Homo sapiens
gtccagtgggacatg

SEQ ID NO: 27 - DNA, 15 bp, Homo sapiens
ttagaaataaactat

SEQ ID NO: 28 - DNA, 15 bp, Homo sapiens
ttagaaatcaactat

SEQ ID NO: 29 - DNA, 15 bp, Homo sapiens
ctggaatcgattata

SEQ ID NO: 30 - DNA, 15 bp, Homo sapiens
ctggaattgattata

SEQ ID NO: 31 - DNA, 42 bp, Homo sapiens
gtctcttgatttccttttttgctttcttgtcatagacctgac

SEQ ID NO: 32 - DNA, 42 bp, Homo sapiens
gtctcttaatttccttttttgctttcttgtcatagacctgac

(19) United States
(12) Patent Application Publication
(10) Pub. No.: US 2004/0081981 A1
(43) Pub. Date: Apr. 29, 2004

(54) Method of detecting risk factor for onset of diabetes

(76) Inventors: Toru Egashira, Kawagoe-shi (JP); Makoto Nagano, Kawagoe-shi (JP); Yukiko Sagehashi, Kawagoe-shi (JP); Kana Matsui, Kawagoe-shi (JP); Hiroaki Hattori, Kawagoe-shi (JP); Azuma Kanatsuka, Chiba-shi (JP); Shin Takasawa, Sendai-shi (JP); Hiroshi Okamoto, Sendai-shi (JP)

Correspondence Address: KNOBBE MARTENS OLSON & BEAR LLP, 2040 MAIN STREET, FOURTEENTH FLOOR, IRVINE, CA, 92614, US

(21) Appl. No.: 10/454,238

(22) Filed: Jun. 4, 2003

Related U.S. Application Data
(62) Division of application No. 09/773,307, filed on Jan. 31, 2001, now abandoned.

(51) Int. Cl. C12Q 1/68

(52) U.S. Cl. 435/6

(57) ABSTRACT

Risk factors for diabetic onset in a subject are detected by using a gene coding for CD38 protein, which is originally identified as a human lymphocyte surface marker, as a risk gene, and detecting mutations in the gene.

### BACKGROUND OF THE INVENTION

1. Field of the Invention

The present invention relates to a method for the detection of a disease. More particularly, the present invention relates to a method for detecting risk factors related to the onset of diabetes in a subject.

2. Description of the Related Art

Today, at least 30 million people worldwide are diabetics, and currently the number is increasing along with a rapid rise in the elderly population. Thus, there is a firm belief that a further increase in the number of diabetic patients will inevitably occur in the near future. Japan is not exempt from this world trend; at present, about 6 million Japanese people are afflicted with diabetes, and in several years the number is expected to reach 10 million.

Diabetes (diabetes mellitus) is a disease that can be described as follows: Glucose is one of the nutrients important for the body, in particular for individual cells. However, when glucose is not incorporated into cells effectively, the glucose accumulates in blood, inducing a high blood glucose concentration. As a result, glucose is excreted into the urine.

Diabetes can be broadly classified into 3 categories: insulin-dependent diabetes mellitus (IDDM; type I diabetes), non-insulin-dependent diabetes mellitus (NIDDM; type II diabetes), and other types of diabetes called secondary diabetes that are incidentally induced by the onset of particular diseases, such as pancreatitis.

Insulin-dependent diabetes (IDDM; type I diabetes) has its onset at a relatively young age. After onset, progress is rapid, resulting in accumulation of metabolites; i.e., ketone bodies, in blood. Without continuous treatment with insulin, the patient's life is endangered. Thus, this type of diabetes is very serious.

In contrast, non-insulin dependent diabetes (NIDDM; type II diabetes) often has its onset after adulthood is reached. The symptoms gradually progress, starting from abnormalities in intravenous glucose tolerance (IGT). Thus, this type of diabetes progresses relatively slowly, and treatment with insulin is not always necessary.

As described above, there are two principal types of diabetes, with two corresponding approaches to treatment. In 1997, the American Diabetes Association (ADA) proposed new classification and diagnosis standards for diabetes. In Japan also, a new concept of diabetes, new classification, and standards for diabetes have been discussed. The new classification is organized on the basis of combinations between the causes of diabetes and symptoms. The symptoms are by and large the same as those mentioned above. On the other hand, now, understanding the causes of diabetes will become more and more important when carrying out diagnosis and classification. As for the new classification, the most noteworthy point is that diabetes caused by genetic abnormalities is incorporated into the third category of “other particular types of diabetes.”

Concerning genes involved in the onset of diabetes, abnormalities in the genes encoding insulin, insulin receptors, glucokinase (MODY2), HNF-4α (MODY1), HNF-1α (MODY3), and mitochondria have so far been known. However, the incidence rates of such abnormalities have been low in all cases. For example, although the mutation from adenosine to guanine at nucleotide position 3243 of the mitochondrial DNA is found at a relatively high incidence rate as compared with other genes involving gene abnormalities, the corresponding mutation is found in less than 1% of the NIDMM cases; and even when the other mutations in the mitochondria are all added together, the total mutation incidence rate is considered to reach a level of about 2%. Thus, it may be concluded that the above-mentioned gene mutations can explain only why a tiny proportion of a large number of diabetic patients contract the condition. In addition, it is generally regarded that diabetic onset is induced by not just a single gene abnormality, but by a multiplicity of gene abnormalities together with the influence of various environmental factors.

Given the facts cited above, examining and determining those genes contributing to the onset of diabetes or risk factors involving the onset is important not only in order to carry out diagnosis of diabetes but also to facilitate the selection of those individuals belonging to the high risk group so that counter measures can be introduced to delay the onset, as well as to find clues which will assist us in understanding the onset mechanisms and thus help us find appropriate therapeutic methods to treat diabetes.

### SUMMARY OF THE INVENTION

Problems to be solved by the present invention are (1) to know what type of diabetes a diabetic individual is presently suffering from; and (2) to find risk genes in order to predict whether or not an individual who presently appears healthy carries risk factors that are capable of causing diabetes in the future, and further to predict what type of diabetes the individual would suffer from in that event (note that hereafter a factor associated with the above (1) and/or (2) will be also referred to as a “risk factor for diabetic onset”); and also to provide means for detecting the risk factors for diabetic onset using the risk genes.

In order to solve the problems, the present inventors have searched risk genes enabling us to determine risk factors for diabetic onset by analysis thereof, and found that abnormalities of the gene coding for CD38 (hereafter the gene will be also referred to as the CD38 gene) induce diabetic onset. CD38 is an enzyme participating in both production and hydrolysis of cyclic ADP ribose (cADPR), a second messenger, capable of stimulating insulin secretion upon glucose stimulation in β cells of Langerhans' islands in the pancreas. Thus, the present inventors concluded that the gene CD38 is the risk gene enabling us to determine risk factors for diabetic onset; i.e., the gene the present inventors have been looking for. The present invention has been accomplished on the basis of the above finding.

Accordingly, the present invention provides a method of detecting genetic factors (risk factors) in relation to diabetic onset in individuals through detection of abnormalities in the gene CD38. The method may hereafter be referred to as the detection method of the present invention.

The CD38 protein encoded by the gene CD38 is a protein originally identified as a human lymphocyte surface marker. The close relationship between the CD38 protein and glucose metabolism in the body has been discovered through long-term studies by the present inventors Okamoto, Takasawa, et al.

### BRIEF DESCRIPTION OF THE DRAWINGS

FIG. 1 shows an abnormal band pattern obtained from gene mutation analysis of exon 2 of the CD38 gene by use of the DGGE method.

FIG. 2 shows an abnormal band pattern obtained from gene mutation analysis of exon 3 of the CD38 gene by use of the DGGE method.

FIG. 3 shows an abnormal band pattern obtained from gene mutation analysis of exon 4 of the CD38 gene by use of the DGGE method.

FIG. 4 shows an abnormal band pattern obtained from gene mutation analysis of exon 7 of the CD38 gene by use of the DGGE method.

FIG. 5 shows an abnormal band pattern obtained from gene mutation analysis of exon 8 of the CD38 gene by use of the DGGE method.

FIG. 6 shows nucleotide sequences identified by direct sequencing for exon 2 of the CD38 gene that exhibited an abnormal band pattern under application of the DGGE method.

FIG. 7 shows nucleotide sequences identified by direct sequencing for exon 3 of the CD38 gene that exhibited one abnormal band pattern under application of the DGGE method.

FIG. 8 shows nucleotide sequences identified by direct sequencing for exon 3 of the CD38 gene that exhibited the other abnormal band pattern under application of the DGGE method.

FIG. 9 shows nucleotide sequences identified by direct sequencing for exon 4 of the CD38 gene that exhibited one abnormal band pattern under application of the DGGE method.

FIG. 10 shows nucleotide sequences identified by direct sequencing for exon 4 of the CD38 gene that exhibited the other abnormal band pattern under application of the DGGE method.

FIG. 11 shows nucleotide sequences identified by direct sequencing for exon 7 of the CD38 gene that exhibited an abnormal band pattern under application of the DGGE method.

FIG. 12 shows nucleotide sequences identified by direct sequencing for exon 8 of the CD38 gene that exhibited an abnormal band pattern under application of the DGGE method.

FIG. 13 shows the results of analysis of the exon 3 Arg140Trp mutation by PCR-RFLP.

FIG. 14 shows the results of analysis of the exon 7 Ser264Leu mutation by PCR-RFLP.

FIG. 15 shows results of analysis of the intron 7-28G/A mutation by PCR-RFLP.

### DETAILED DESCRIPTION OF THE PREFERRED EMBODIMENTS

Before describing a variety of modes of the present invention, below, the relationships between the key player in the present invention; i.e., the gene CD38 or the CD38 protein, and glucose metabolism in the body will first be summarized.

Glucose is the most important substance for stimulating insulin secretion from β cells of the Langerhans' islands in the pancreas. Concerning insulin secretion, it is generally regarded that upon the glucose stimulation, the intracellular Ca²⁺ concentration is elevated to trigger insulin secretion. The increase in the intracellular Ca²⁺ level is accomplished by both the influx of extracellular Ca²⁺ into cells and recruitment from the intracellular Ca²⁺ pool. Concerning the influx of extracellular Ca²+, the theory of “ATP-sensitive K+channel” proposed by Ashcroft et al. is widely accepted. As for the molecular mechanisms, its detailed explanations have been provided by Inagaki, Kiyono, et al. (Seikagaku, 69: 1067-1080, 1997).

In contrast, as for the recruitment of Ca²⁺ from the intracellular Ca²⁺ pool, it has been pointed out that the increase in the intracellular Ca²⁺ level occurs at the same time or earlier than does the influx of extracellular Ca²⁺ from an experiment with human insulin-producing cells (Rojas E. et al., Endocrinology, 134: 1771-1781, 1994). Further, it has been reported that inositol 1, 4, 5-triphosphate (IP₃) functions as a second messenger for the Ca²⁺ recruitment in many types of cells. Similarly, it has been suggested that IP₃ plays an important role as a second messenger for Ca²⁺ recruitment in the insulin-producing cells.

The present inventors Okamoto et al. demonstrated that maintaining the intracellular NAD⁺ level in β cells is a prerequisite for maintaining the insulin-producing function of β cells, showing that DNA damage is induced by the action of a cytotoxic agent for insulin-producing β cells, such as streptozotocin, and that, as a result, the cytotoxic agent causing DNA damage induces activation of poly (ADP-ribose) synthetase, which in turn causes intracellular NAD⁺ to be depleted, resulting in lowering the insulin-producing ability of β cells. In contrast, as they have shown, when lowering of the intracellular NAD⁺ concentration is inhibited by blocking with an inhibitor of poly (ADP-ribose) synthetase, insulin-producing ability of β cells is maintained (Yamamoto H, et al., Nature, 294: 284-286, 1981; Okamoto H, et al., 1990, in Molecular Biology of the Islands of Langerhans, Okamoto H, ed. pp. 209-231, Cambridge University Press, Cambridge; Okamoto H, et al., Biochimie, 77: 356-363, 1995). Based on this background, concerning the mechanisms of insulin secretion involving glucose, the present inventors Okamoto, Takasawa, et al. have suggested that cADPR synthesized from NAD⁺ is involved in expression of the insulin-producing cell function.

In the case of Ca²⁺ releasing experiments with Fluo3 using microsomes prepared from the Langerhans' islands of rat pancreases, cADPR induces the Ca²⁺ release from the microsomes of Langerhans' islands, and continuous addition of cADPR confirms attenuation of Ca²⁺ release. In contrast, upon addition of IP₃, Ca²⁺ release is not observed. From these results, the present inventors Takasawa, Okamoto, et al. have concluded that the Ca²⁺ release with cADPR from the Langerhans' islands microsomes differs from that with IP₃ (Takasawa S, et al., Science, 259: 370-373, 1993). Further, from radioimmunoassays with an antibody against cADPR, Takasawa, Okamoto et al. showed that the amount of cADPR in insulin-producing cells is increased upon glucose stimulation (Takasawa S, et al., J. Biol. Chem., 273: 2497-2500, 1998). From these results, Ca²⁺ recruitment upon glucose stimulation observed in the insulin-producing cells is considered to be due mainly to cADPR.

Further, Takasawa, Okamoto, et al. demonstrated that the CD38 protein shows ADP-ribosyl cyclase activity capable of synthesizing cADPR when NAD⁺ is used as a substrate, and on the other hand, when cADPR is used as a substrate, the CD38 protein shows cADPR hydrolase activity capable of synthesizing ADPR (Takasawa S, et al., J. Biol. Chem., 268: 26052-26054, 1993). It is well known that the cADPR hydrolysis reaction is suppressed with 2-8 mM ATP in a concentration-dependent manner, whereas, when NAD⁺ is used as a substrate, the amount of cADPR production is increased in an ATP concentration-dependent manner. Further, Tohgo, Takasawa et al. demonstrated the molecular mechanism of suppression of cADPR hydrolase activity by ATP is due to competition between ATP on the order of mmol units and cADPR for the cADPR binding site at residue 129Lys of the CD38 protein (Tohgo A, Takasawa S, et al., J. Biol. Chem., 272: 3879-3882, 1997). In fact, it has been reported that the ATP concentration in the insulin-producing cells is changed within a range of 2-8 mM upon glucose stimulation, and further the change of cADPR amount could be explained by the mechanism that cADPR hydrolysis activity of the CD38 protein is suppressed by ATP, assuming that the CD38 protein is present in the insulin-producing cells.

Further, together with Kato and others, Takasawa, Okamoto, et al. have demonstrated that in transgenic mice overexpressing the CD38 protein in β cells of the Langerhans' islands, the insulin secretion in response to glucose is facilitated significantly as compared with the control mice (J. Biol. Chem. 270, 30045-30050, 1995).

As described above, the mechanism of insulin secretion is considered as follows: cADPR hydrolysis activity of the CD38 protein is suppressed by ATP, which is produced by glucose stimulation, resulting in an increase in the cADPR amount, followed by the Ca²⁺ recruitment from the intracellular Ca²⁺ pool in microsomes, thereby causing insulin secretion.

Further, the Ca²⁺ release mechanism by cADPR is considered to be through ryanodine receptor (type 2) in endoplasmic reticulum, since the Ca²⁺ release with cADPR is cross-desensitized with Ca²⁺ release with ryanodine, as discovered by Okamoto, Takasawa, et al. Takasawa, Okamoto, et al. demonstrated that, in the Langerhans' β cells, Ca²+/calmodulin(CaM)-dependent protein kinase II phosphorylates ryanodine-type Ca²+-releasing channels (i.e. cADPR-sensitive Ca²⁺-releasing channels) in endoplasmic reticulum. Due to the phosphorylation, the Ca²⁺-releasing sensitivity to cADPR is enhanced to elicit release of Ca²⁺ through the sensitized ryanodine-type Ca²⁺-releasing channels upon glucose stimulation (J. Biol. Chem. 270: 30257-30260, 1995). Further, Takasawa, Okamoto, et al. demonstrated that the Ca²⁺-releasing with IP₃ dominantly occurs in ob/ob mice, whereas the Ca²⁺-releasing with cADPR mainly occurs in normal mice (Takasawa S, et al., J. Biol. Chem., 273: 2497-2500, 1998). Furthermore, it has also been suggested that exhaustion and consumption of intracellular NAD⁺ in insulin production cause diabetes, since it has been reported that knock-out mice lacking the poly (ADP-ribose) polymerase (PARP) gene generated by gene-targeting show resistance to the onset of diabetes to be induced with streptozotocin (Burkart V, et al., Nature Med., 5: 314-319, 1999). In conclusion, Okamoto, Takasawa, et al. demonstrated that the process involving cADPR production using the NAD⁺ substrate, recruitment of intracellular Ca²⁺, and insulin secretion is important for insulin-producing cells, and that the CD38 gene or CD38 protein is intimately involved in the process. Thus, from our results and analyses, the present inventors have come up to the possibility that abnormalities of the gene CD38 could be used to detect risk factors for diabetic onset. The gene is closely involved in the onset, however, it should be pointed out that this deduced possibility was very surprising in consideration that all the genes whose abnormalities were reportedly associated with diabetes have not always turned out to be clinically useful as markers for detecting risk factors for diabetic onset.

The major sites of abnormality of the gene CD38 that can be used for the detection method in the present invention are as follows: (1) the site encoding arginine at amino acid residue 140 of the CD38 protein encoded by the CD38 gene; (2) the site encoding serine at amino acid residue 264; and (3) guanine at nucleotide position −28 in intron 7. Note that abnormalities at these sites are independent from each other and are not related. More details of these sites with genetic abnormalities will be described later in the Examples section.

A variety of modes of the present invention will next be described.

As described above, the present invention is directed to a method of detecting risk factors for diabetic onset on the basis of detection of genetic abnormalities of the gene CD38, a risk gene concerning diabetic onset.

The CD38 gene and its encoding CD38 protein have already been analyzed [Nata K. et al., Gene, 186: 285-292, 1997; Sequence No. 1 (nucleotide and amino acid sequences) and Sequence No. 2 (amino acid sequence)].

By examining the correlation between genetic mutations of the CD38 gene and risk factors for diabetic onset, genetic mutations of the CD38 gene useful for the detection method of the present invention can be found. For this, desirable mutations utilized for the present invention can be found by analyzing combinations chosen between “diabetic patients and healthy individuals” and between “insulin-dependent diabetes and non-insulin dependent diabetes” concerning mutation sites of the CD38 gene, their incidences, and functions of their encoding proteins. Detailed working protocols will be described hereinbelow in the Examples section.

As used herein, “genetic mutation” refers to mutation of a human chromosomal gene, the nucleotide sequence of which differs from a wild-type nucleotide sequence (a nucleotide sequence of a normal gene). Further, although particular nucleotide sequence sites different from person to person are generally regarded as “genetic polymorphism,” in the present invention these are classified as “genetic mutation.”“Genetic mutation” is identified by analyzing various aspects such as the incidence rate of gene mutation, expression levels of its mRNA and protein, or its protein function. It is generally regarded that, out of several hundreds of nucleotides, on average one such “genetic mutation” occurs; and such a mutation can be identified through direct or indirect analysis of the gene. Further, through analysis of the involved family trees, whether such a genetic mutation is derived from the paternal chromosome allele or the maternal chromosome allele can also be identified.

In the case of changes in a genetic mutation site, substitution of a nucleotide base takes place at the corresponding site of either the paternal allele and/or the maternal allele. When the nucleotide bases of both alleles are substituted and differ from the wild-type, it is called a “homozygote,” and when the nucleotide base of only one allele is substituted and differs from the wild-type, it is called a “heterozygote.”

As will be described later, the present inventors have so far found the following genetic mutation sites correlated with risk factors for diabetic onset: (1) the site encoding arginine at amino acid residue 140 of the CD38 protein encoded by the CD38 gene (for example, the mutation is determined by the sensitivity of the exon 3 region to a restriction enzyme TspRI.); (2) the site encoding serine at amino acid residue 264 (for example, the mutation is determined by the sensitivity of the exon 7 region to a restriction enzyme TaqI.); and (3) the site encoding guanine at nucleotide position −28 in intron 7 (for example, the mutation is determined by the sensitivity of a region containing an intron 7 and exon 8 region to a restriction enzyme Tru9I.).

Concerning the detection methods of abnormal mutation sites, generally known methods can be used; for example, the RFLP method using Southern blotting, the PCR-RFLP method, the HET (heteroduplex analysis) method, the DGGE (denaturing gradient gel electrophoresis) method, the DS (direct sequencing) method, the CCM (chemical cleavage mismatch) method, the CDI (carbodiimide modification) method, the PCR-SSCP (single-stranded conformation polymorphism) method using the PCR method, which in the present specification is hereafter referred to as the SSCP method, and the PCR/GC-clamp method (for details, see, for example, Bio-Manual series I: “Basic Techniques for Gene Engineering,” Tadashi Yamamoto, ed., Yodosha, (1993); in particular, concerning the PCR/GC-clamp method, for example, see Myers, R. M., Shefield, V., and Cox, D. R. (1988) in Genomic Analysis: A Practical Approach. K. Davies, ed. IRL Press Limited, Oxford, pp. 95-139). Among the above detection methods, the choice of the PCR/GC-clamp method is preferred because of its easy handling and determination accuracy of genetic abnormalities.

PCR/GC-clamp method is a modified method of the DGGE method (which detects DNA nucleotide substitution based on a migration difference between a double-stranded DNA fragment with a substituted base(s) and that without a substituted base, since they migrate differently due to their different migration speeds varying respectively depending on concentrations of a DNA-denaturation agent; in this case, on a linear concentration gradient of DNA-denaturation agent contained in a polyacrylamide gel). However, the DGGE method has a drawback in that “in the case of the presence of substituted multiple bases in the testing DNA fragments, the base substitution in a domain to be melted last in a polyacrylamide gel cannot be detected.” In contrast, PCR/GC-clamp method has overcome this problem by ligating a high GC-rich region (GC-clamp) to the testing DNA fragment (see Shefield, V. C. et al., (1989) Proc. Natl. Acad. Sci. USA 86: 232-236).

Thus, the operation of the PCR/GC-clamp method is basically the same as that of the DGGE method (as is the case with the PCR/GC-clamp method,.see Myers, R. M., Shefield, V., and Cox, D. R. (1988) in Genomic Analysis: A Practical Approach. K. Davies, ed. IRL Press Limited, Oxford, pp. 95-139), except that there is an additional step; i.e. ligating the GC-clamp to the testing DNA sample, required for detection of base substitution.

The DNA sources needed in order to detect changes at particular sites carrying a possible genetic abnormality of the CD38 gene in the method of the present invention are not particularly limited, so long as they are derived from somatic cells of subjects. For example, blood samples such as peripheral blood or leukocytes can be successfully used for the method of the present invention.

Genomic DNA is extracted from testing cells of individual subjects using a conventional method(s). By use of the extracted genomic DNA, changes at particular sites carrying a possible genetic abnormality (to be precise, base substitution at particular sites with a possible genetic abnormality) are detected.

Then, when such changes are detected, in consideration of the changes in combination with clinical symptoms of the subjects, the risk factors for diabetic onset of the subject can be determined.

That is, in the case of the subject already experiencing diabetic onset, the major cause for diabetic onset can be identified. Further, if the symptoms indicate non-insulin dependent diabetes, whether or not the diabetes will proceed to insulin-dependent diabetes can be predicted. As a result, treatments suitable for the symptoms or preventive measures can be provided, or the optimum treatment method can be developed.

Alternatively, in the case of the subject who has not yet experienced diabetic onset or who has been determined to belong to the risk group on the basis of results of a different examination or family medical history, proper preventive measures against the onset of diabetes, such as practical guidance for diet and exercise, can be provided by regarding changes at particular sites carrying a possible genetic abnormality as a genetic diabetes constitution of the subject. In this way, diabetic onset in the subject may be prevented.

### EXAMPLES

The present invention will next be described in detail by way of examples, which should not be construed as limiting the present invention.

Analysis of the CD38 Gene

Subjects

Mutation analysis of the CD38 gene was carried out with the DGGE method using 240 subjects who had been diagnosed as diabetes based on the diagnosis criteria proposed by the Japan Diabetes Society.

Extraction of Genomic DNA

After collecting peripheral blood from the diabetic patients using blood-collecting vacuum tubes containing an anti-coagulation agent EDTA 3K, genomic DNA from healthy persons and the diabetic subjects were respectively extracted by use of a QIAamp Blood Kit (QIAGEN).

Set-Up of PCR Primers and Electrophoresis Conditions for the DGGE Analysis

DNA sequence of the CD38 gene (Sequence No. 1) encoding the human CD38 protein, ADP-ribosyl cyclase/cyclic ADP-ribose hydrolase, was obtained from DDBJ/EMBL/GenBank databases (ACCESSION D84278-84284; Nata, K., et al.).

Primers for amplifying each exon of the CD38 gene using the PCR method were prepared by use of computer software GENETYX-MAC (Software Developing Co. Ltd.). Characteristics (domain positions, melting temperatures, etc.) of melting domains of a DNA-fragment amplified with the PCR primers were predicted by use of computer software Mac Melt (Nippon Bio-Rad Laboratories K.K.). To which primers a GC-clamp should be ligated was predicted from the melting characteristics obtained by computation, and further an optimal concentration range of a denaturation agent suitable for analysis of the resultant PCR DNA fragment was determined.

The nucleotide sequences of PCR primers used for amplification of respective exons of the CD38 gene are shown below:

| | |
|---|---|
| Exon 1 | |
| 5′-CGC CCG CCG CGC CCC GCG CCC GTC CCG CCG CCC CCG CCC GAT CTT | (Forward: Sequence No. 3) |
| CGC CCA GCC AAC CCC G-3′ | |
| 5′-ACC GGT GCG CCT TAG TCG CCA-3′ | (Reverse: Sequence No. 4) |
| Exon 2 | |
| 5′-TAG ACT GCA TGT TAG ACG AGA-3′ | (Forward: Sequence No. 5) |
| 5′-CGC CCG CCG CCC CCC GCG CCC GTC CCG CCG CCC CCG CCC GTT TGG | (Reverse: Sequence No. 6) |
| ACC TAT GAA TTG TTA CC-3′ | |
| Exon 3 | |
| 5′-GAC ATG CTA AAT TGA TCT CAG-3′ | (Forward: Sequence No. 7) |
| 5′-CGC CCG CCG CCC CCC GCG CCC CTC CCG CCG CCC CCG CCC GCA GCA | (Reverse: Sequence No. 8) |
| GAA GTC ACT CTG TTC-3′ | |
| Exon 4 | |
| 5′-CCA TTC TCC AGC CTC CGT CTT-3′ | (Forward: Sequence No. 9) |
| 5′-CGC CCG CCC CGC CCC CCG CCC GTC CCC CCG CCC CCG CCC GCA AGC | (Reverse: Sequence No. 10) |
| ACT GAC TGA OTA ACO TC-3′ | |
| Exon 5 | |
| 5′-AAA CTG CTG GAO GAT GGT GAT T-3′ | (Forward: Sequence No. 11) |
| 5′-CGC CCC CCG CGC CCC GCG CCC GTC CCG CCC CCC CCG CCC GTT CAC | (Reverse: Sequence No. 12) |
| TGT GAT ATT TGC AAC AGG-3′ | |
| Exon 6 | |
| 5′-GGT TGA TGT TTG GGG TTC TTT GT-3′ | (Forward: Sequence No. 13) |
| 5′-CGC CCC CCC CGC CCC GCG CCC GTC CCG CCG CCC CCG CCC GTG TGG | (Reverse: Sequence No. 14) |
| ATT CTT TTG TGG ACT GAT T-3′ | |
| Exon 7 | |
| 5′-CCC CCG CCG CGC CCC GCG CCC GTC CCG CCG CCC CCG CCC GTT GTC | (Forward: Sequence No. 15) |
| CAG GGC GTG CTA CAA A-3′ | |
| 5′-AGA TTC ACA CAG CCC TCC AAG-3′ | (Reverse: Sequence No.16) |
| Exon 8 | |
| 5′-CGC GCG CCG CGC CCC GGG CCC GTG CCC CCG CCC CCC CCC GTT AGC | (Forward: Sequence No. 17) |
| GAA TTG GAG GAG AGA TG-3′ | |
| 5-TCT GGC ATT GAG GTT ATT GTG G-3′ | (Reverse: Sequence No. 18) |
| Exon 3 | |
| 5′-CTC CGC GAG TGT GGT GGA GAG A-3′ | (Forward: Sequence No. 19) |
| 5′-GGG GGT GGA GGA GAA GTG AC-3′ | (Reverse: Sequence No. 20) |
| Exon 7 | |
| 5′-TTG TGG AGG GGG TGC TAG AAA-3′ | (Forward: Sequence No. 21) |

PCR Amplification

By use of 0.5 μg/μL extracted genomic DNA, PCR amplification of each exon was carried out in a 50 μL PCR reaction mixture [10 mM Tris-HCl buffer (pH 8.3), 50 mM KCl, 1.5 mM MgCl₂] which contained 0.8 μM synthetic oligonucleotide primer for each exon, 200 μM nucleotide triphosphate (dNTP) and 3% formamide and to which 0.5 units of Taq DNA polymerase (Perkin Elmer) had been added under the following amplification conditions: 40 cycles of a series of reactions at 94° C. for 30 sec, at 54-68° C. for 30 sec, and at 72° C. for 1 min, followed by the last 10 min reaction at 72° C., to thereby obtain PCR products. Confirmation of the PCR reaction was carried out by performing 3% agarose gel electrophoresis using each 5 μL PCR product followed by staining with 0.5 μg/mL ethidium bromide.

Note that each exon of the CD38 gene corresponds to the following nucleotide sequence positions: exon 1, nucleotide positions 1-233 of Sequence No. 1; exon 2, 234-363 of Sequence No. 1; exon 3, 364-498 of Sequence No. 1; exon 4, 499-585 of Sequence No. 1; exon 5, 586-659 of Sequence No. 1; exon 6, 660-752 of Sequence No. 1; exon 7, 753-839 of Sequence No. 1; and exon 8, 840-890 of Sequence No. 1.

Screening of Genetic Mutations by the DGGE Method

DGGE method was carried out by following the protocol designed by Myers, et al. The denaturing gradient polyacrylamide gel was prepared by use of glass plates (177×220 mm) (Takara Shuzo, Co. Ltd.). The optimal concentration of a denaturing agent was chosen for each exon, and each concentration gradient was designed to be within a range of 30%. Each polyacrylamide gel containing an optimal concentration gradient of a denaturing agent was prepared by a gradient maker, mixing 9% acrylamide (acrylamide: bis-acrylamide=37.5:1) in TAE solution (40 mM Tris, 20 mM sodium acetate, and 2 mM EDTA (pH 7.4)) containing 0% denaturing agent and 9% acrylamide in TAE solution containing 80% denaturing agent (5.19 M urea and.30% deionized formamide).

Screening genetic mutations of the PCR products by the DGGE method was carried out as follows: after aliquoting 15 μL of the above-mentioned PCR product from each exon to a 500-μL tube, the sample was dried in a vacuum dryer; to each sample tube, 10 μL loading buffer [20% Ficoll, 1 mM EDTA, and 0.5% bromophenol blue in 10 mM Tris-HCl buffer (pH 7.8)] was added; after dissolving, 5 μL sample was each applied to the gel. Electrophoresis was carried out under conditions of 150 V×16 h in a DDGE electrophoretic chamber (Takara Shuzo, Co. Ltd.) containing 1×TAE solution (16 L) kept at 60° C. After electrophoresis, the gel was subjected to staining with 0.5 μg/mL ethidium bromide, and then DNA was visualized under UV light, followed by pattern analysis.

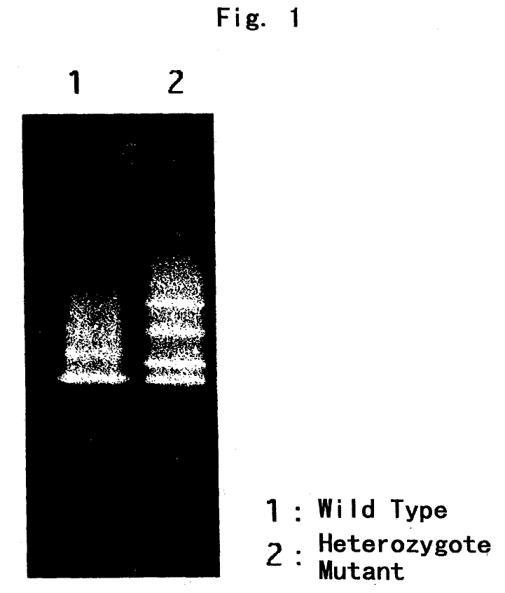

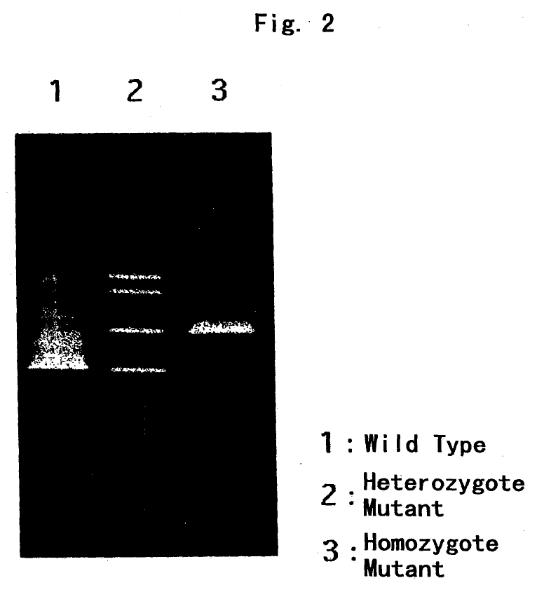

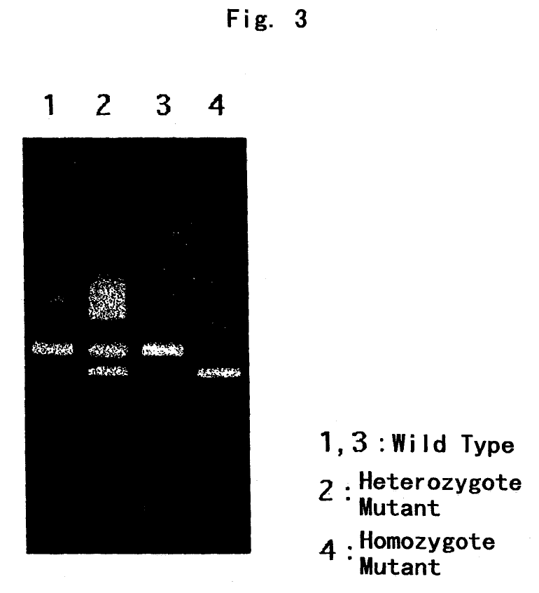

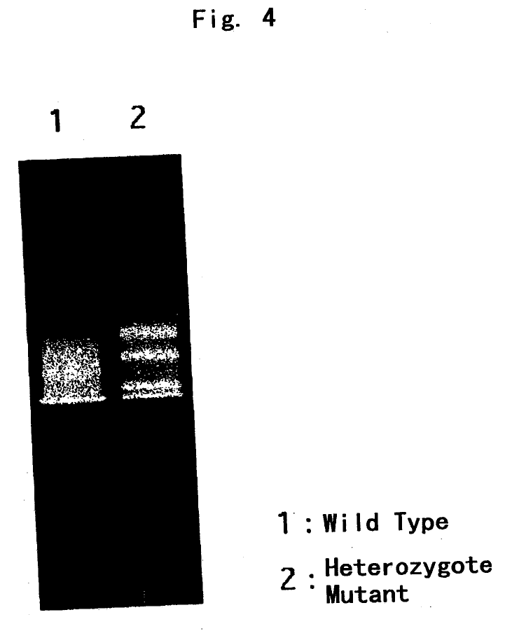

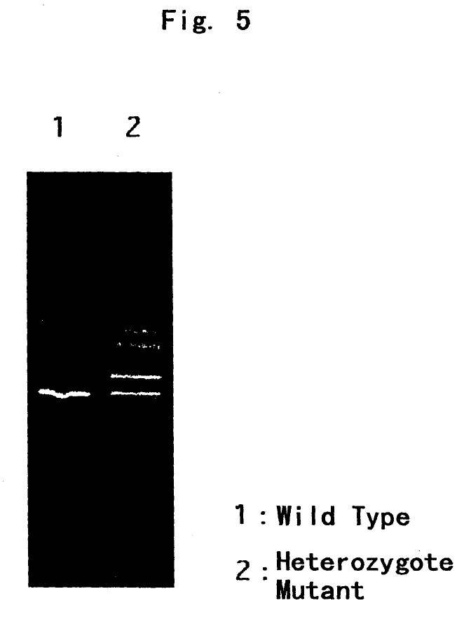

The results obtained by screening mutation in each exon and intron region of CD38 genes using the DGGE method showed that there were band patterns differing from the wild type found 1 at exon 2 (FIG. 1), 2 at exon 3 (FIG. 2), 2 at exon 4 (FIG. 3), 1 at exon 7 (FIG. 4), and 1 at exon 8 (FIG. 5). These were confirmed to be abnormal band patterns. Concerning the gene abnormality, the obtained abnormal band patterns demonstrated by the DGGE method suggest that the corresponding PCR products contain nucleotide sequences different from the wild type. Determination of nucleotide sequences thereof would have to be carried out by another method.

Determination of Nucleotide Sequences by Direct Sequencing

The PCR products with abnormal patterns shown by the DGGE method were subjected to DNA sequencing with an automated sequencer using a direct sequencing method. The PCR products showing the abnormal patterns were fluorescence-labeled by use of BigDye Terminator cycle sequencing Fs Ready Reaction Kit (Perkin Elmer). Then, the samples were subjected to nucleotide sequencing by use of ABI PRISM 377 DNA sequencer (Applied Biosystems).

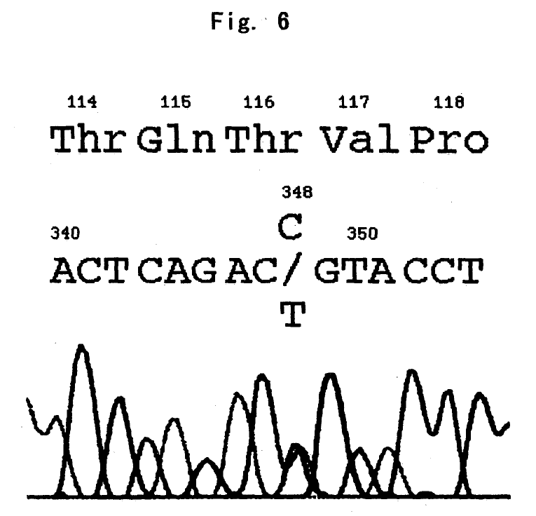

The direct sequencing results obtained on an automated sequencer were subjected to analysis, showing that the individual exhibiting the abnormal band pattern for exon 2 carried thymine (T) besides the wild-type cytosine (C) at nucleotide position 348. Thus, the base substitution of C with T was identified at nucleotide position 348 (FIG. 6). Consequently, due to the single base substitution from C to T, the corresponding codon at 116 amino acid residue was changed from ACC to ACT. However, the encoded amino acid residue was the same threonine (Thr), resulting in no amino acid substitution due to the single base substitution. Thus, the base substitution from C to T at nucleotide position 348 turned out to be not accompanied with amino acid substitution, a so-called silent mutation, and the individual exhibiting the abnormal pattern carried the heterozygote with C348T mutation.

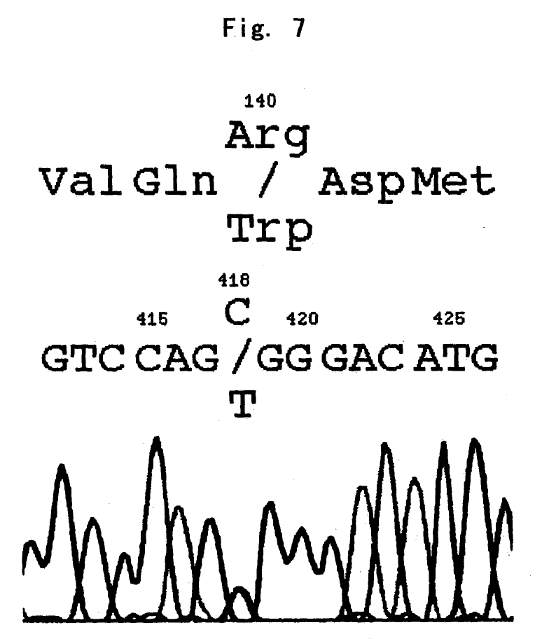

One of individuals exhibiting an abnormal pattern for exon 3 showed T at nucleotide position 418 in addition to the wild-type C. Due to the one-base substitution from C to T, the corresponding codon at amino acid residue 140 was converted from CGG to TGG, resulting in the change at amino acid residue 140 from arginine (Arg) to tryptophan (Trp), which is shown in FIG. 7. Thus, the individual with the abnormal band pattern for exon 3 carried one amino acid substitution Arg140Trp at amino acid residue 140, a so-called missense mutation, and carried the heterozygote with Arg140Trp mutation.

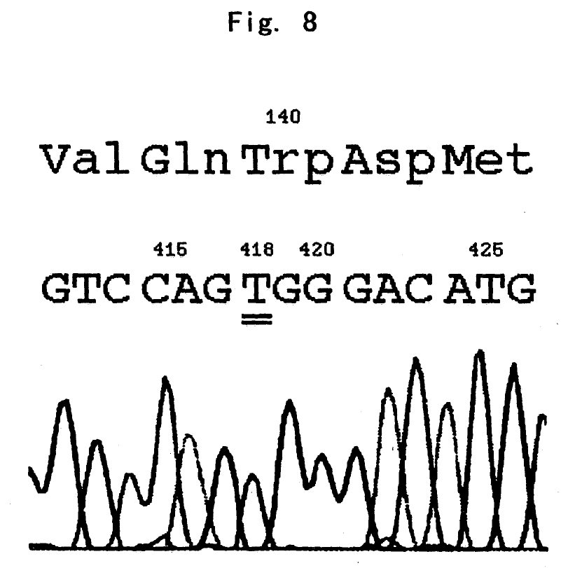

Sequencing of DNA from the other individual exhibiting an abnormal pattern for exon 3 showed only T base at nucleotide position 418, and the individual turned out to carry the homozygote with Arg140Trp mutation (FIG. 8).

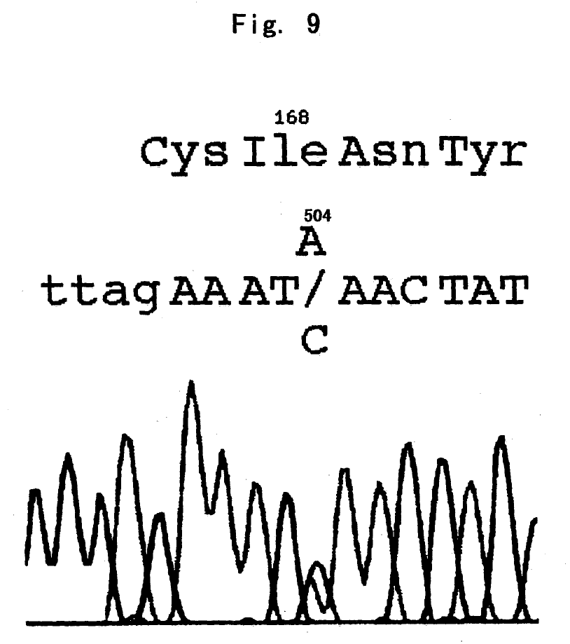

One of individuals with an abnormal pattern for exon 4 showed C at nucleotide position 504 in addition to the wild-type A. Due to one-base substitution from A to C, the corresponding codon at amino acid residue 168 was changed from ATA to ATC, as shown in FIG. 9. However, the corresponding amino acid at amino acid residue 168 remained the same as isoleucine (Ile). Thus, the one-base substitution from A to C at nucleotide position 504 belonged to the silent mutation, and the individual with the abnormal pattern for exon 4 carried the heterozygote with A504C mutation.

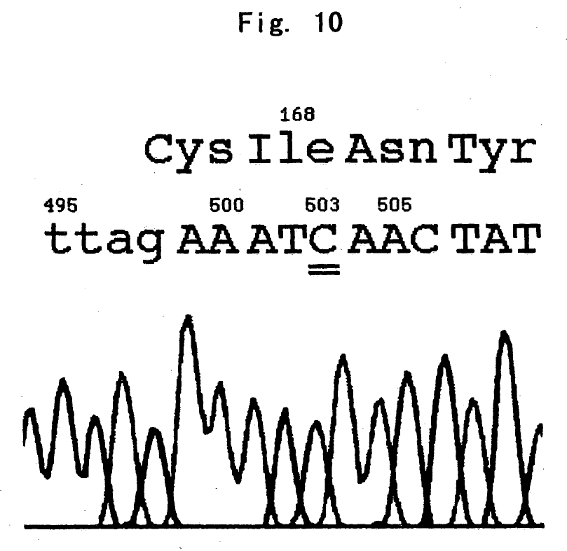

Another individual with an abnormal pattern for exon 4 showed only C at nucleotide position 504, thus carrying the homozygote with A504C mutation (FIG. 10).

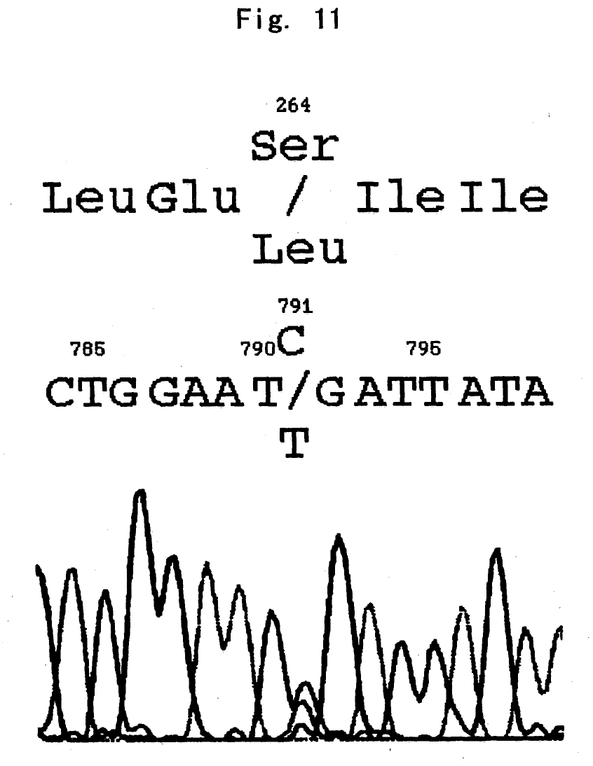

An individual exhibiting an abnormal pattern for exon 7 showed T in addition to the wild-type C at nucleotide position 791. Due to the one base substitution from C to T, the corresponding codon at amino acid residue 264 was changed from TCG to TTG, resulting in one amino acid substitution from serine (Ser) to leucine (Leu) (FIG. 11). Thus, the individual exhibiting the abnormal pattern for exon 7 carried the heterozygote with the amino acid substitution of Ser264Leu at amino acid residue 264 due to one-base substitution at nucleotide position 791 from C to T.

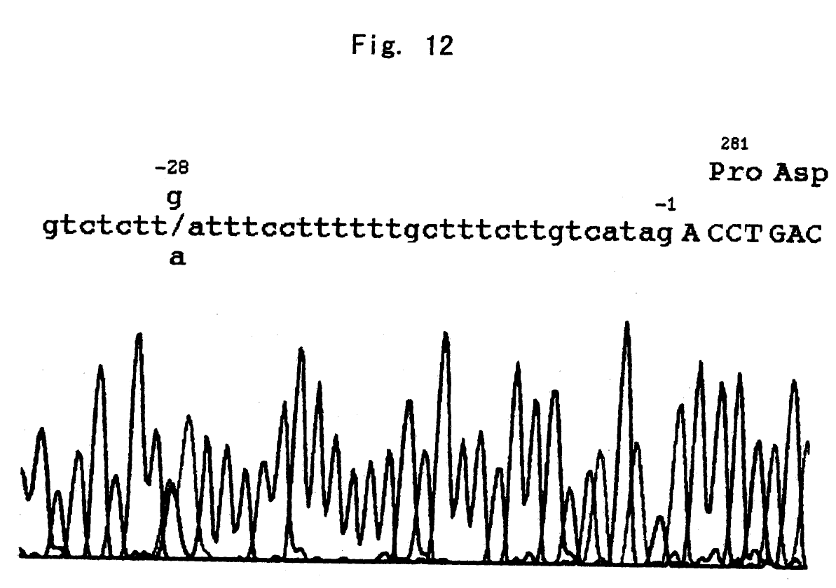

An individual exhibiting an abnormal pattern for exon 8 showed A in addition to wild-type G within intron 7 upstream of exon 8 of the CD gene, at −28 base position from the splicing acceptor site of exon 8 (Sequence No. 22: base position 39 of the intron 7 sequence) (FIG. 12). Thus, the abnormal pattern detected with exon 8 was due to one-base substitution from G to A at base position −28 upstream of the above-mentioned acceptor site, resulting in the heterozygote with G/A mutation at −28 base position within intron 7.

As described above, concerning genetic mutations of the CD38 gene detected in the diabetes group, there were two missense mutations; i.e., Arg140Trp at exon 3 and Ser264Leu at exon 7. In both cases, the corresponding amino acid residues were changed, thus likely causing functional abnormalities in the CD38 protein. Indeed, the CD38 protein carrying Arg140Trp mutation showed lower activity of both ADP-ribosyl cyclase and cyclic ADP-ribose (cADPR) hydrolase (Diabetologia 41: 1024-1028, 1998).

In contrast, in the case of Ser264Leu mutation, it is known that a region consisting of several amino acid residues including the amino acid residue 264 plays an important role in binding to the substrate NAD⁺ upon expression of enzyme activity of the CD38 protein. Thus, the CD38 protein carrying an amino acid substitution at any amino acid residue within the region including the above amino acid residue 264 is expected to have lower enzymatic activity or lose the activity.

The −28G/A mutation at intron 7 appears to be present within a consensus sequence of the so-called “branched site,” that is involved in formation of the lariat structure upon mRNA splicing [Gene (the last volume), Tokyo Kagaku Dojin]. Thus, the −28G/A mutation at intron 7 likely affects generation of CD38 mRNA, and thereby could result in synthesis of a CD38 abnormal protein or abnormal mRNA of the CD38 gene.

Analysis of Incidence Rates of CD38 Gene Mutations

The present inventors have carried out comparative studies of mutation incidence rates between the diabetic and healthy groups using the above-described mutations of the CD38 gene; i.e., C348T mutation at exon 2, Arg140Trp at exon 3, A504C at exon 4, Ser264Leu at exon 7, and the −28G/A mutation at intron 7. Note that in the case of three mutations, Arg140Trp at exon 3, Ser264Leu at exon 7, and the −28G/A mutation at intron 7, sequences recognized by restriction enzymes were changed due to one-base substitution. Thus, to detect the mutations, the PCR-RFLP method was used. In the case of C348T mutation at exon 2 and A504C at exon 4, the PCR-DGGE method was employed, as described above.

Gene Mutation Analysis by the PCR-RFLP Method

Detection Method for Exon 3 Arg140Trp

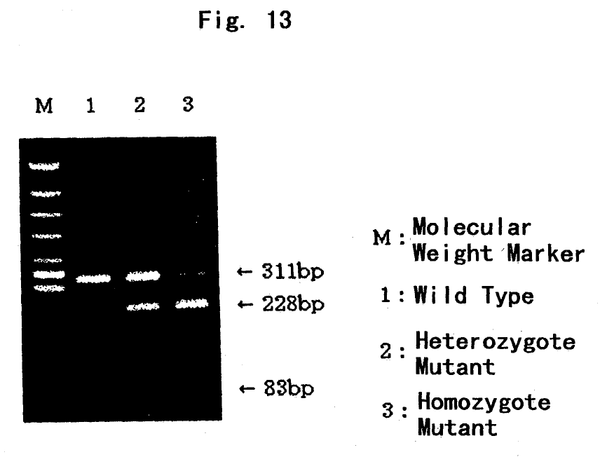

Using 0.5 μL extracted genomic DNA and oligonucleotide primers of Sequence Nos. 19 and 20, PCR reactions were carried out to obtain a PCR product of 381-bp fragment, as described above. After digestion of 10 μL of the resultant PCR product with 2.5 units TspRI restriction enzyme (New England BioLabs) at 65° C. overnight, the digested sample was subjected to 3% agarose gel electrophoresis to detect Arg140Trp at exon 3. The wild-type 381-bp DNA fragment was digested into 2 fragments of 311 and 70 bp, whereas the DNA fragment carrying Arg140Trp mutation was split into 3 fragments of 228, 83, and 70 bp (FIG. 13). In this way, the presence of the mutation was detected.

Detection Method for Exon 7 Ser264Leu

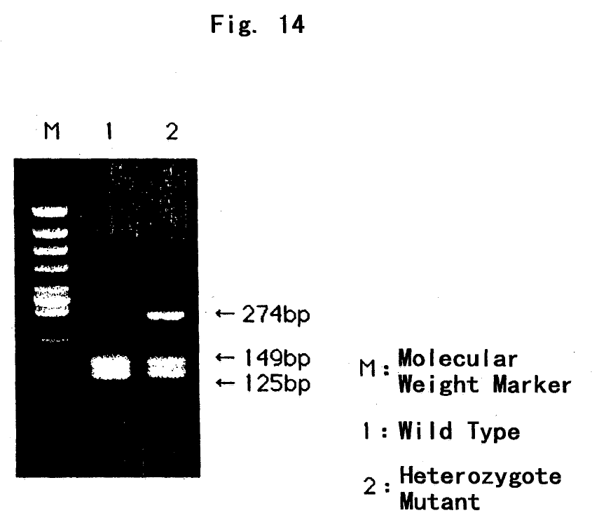

By use of 0.5 μL extracted genomic DNA, oligonucleotide primers of Sequence Nos. 17 and 21, and PCR solution containing the final concentration of 3% formamide, PCR reactions were carried out to obtain a PCR product of 274-bp fragment, as described above. After digestion of 10 μL of the resultant PCR product with 5 units TaqI restriction enzyme (New England BioLabs) at 65° C. overnight, the digested sample was subjected to 3% agarose gel electrophoresis to detect the exon 7 Ser264Leu mutation. The wild-type 274-bp DNA fragment was digested into 2 fragments of 149 and 125 bp, whereas the DNA fragment carrying Ser264Leu mutation was not digested (FIG. 14). In this way, the presence of the mutation was detected.

Detection Method for Intron 7 −28G/A Mutation

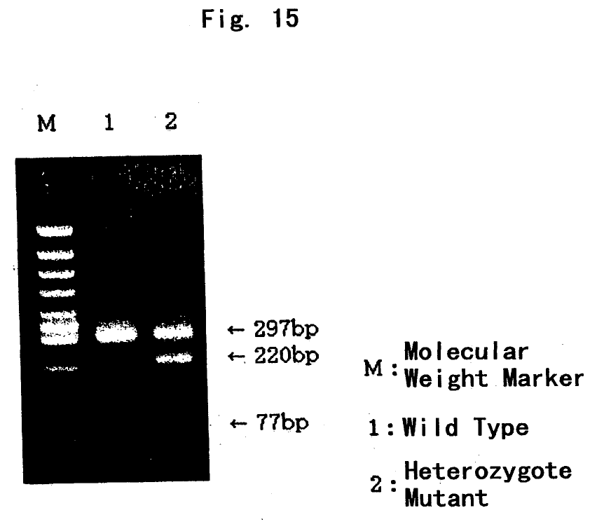

Using 0.5 μL extracted genomic DNA, oligonucleotide primers of Sequence Nos. 17 and 18, and PCR solution containing the final concentration of 3% formamide, PCR reactions were carried out to obtain a PCR product of 297-bp fragment, as described above. After digestion of 10 μL of the resultant PCR product with 2 units Tru9I restriction enzyme (Promega) at 65° C. overnight, the digested sample was subjected to 3% agarose gel electrophoresis to detect the intron 7 −28G/A mutation. The wild-type 297-bp DNA fragment was not digested at all, whereas the DNA fragment carrying the intron 7 −28G/A mutation was digested into 2 fragments of 220 and 77 bp (FIG. 15). In this way, the presence of the mutation was detected.

Incidence Rate of Each Genetic Mutation

The present inventors have analyzed a total of 757 diabetic patients, including 240 cases analyzed genetically by the DGGE method.

The exon 2 C348T mutation was found in 2 out of 240 cases, and its incidence rate was 0.8%. In the case of the exon 3 Arg140Trp mutation, the heterozygote was observed in 27 out of 757 cases, and the homozygote was found in 1 out of 757 cases. Thus, the total incidence rate was 3.7%. In the case of the exon 4 A504C mutation, the heterozygote was found in 58 out of 240 cases, and the homozygote was found in 4 out of 240 cases, showing the total incidence rate of 25.8%. For the exon 7 Ser264Leu mutation, the heterozygote was found in 9 out of 757 cases, showing the incidence rate of 1.2%. For the intron 7 −28G/A mutation, the-heterozygote was found in 9 out of 757 cases, showing the incidence rate of 1.2%.

In contrast, when 205 non-diabetic subjects were analyzed, 3 cases showed the heterozygote for the exon 3 Arg140Trp mutation (3/205 cases), 2 cases showed the heterozygote for the intron 7 −28G/A mutation (2/205 cases), and 0 cases showed the exon 7 Ser264Leu mutation (0/205 cases).

In 2 cases showing the heterozygote for the exon 2 C348T mutation, one case showed the heterozygote for the A504C mutation, whereas the other showed no such A504C mutation but rather the wild-type base sequence. Moreover, out of 58 cases showing the heterozygote for the A504C mutation, 57 cases did not carry any other mutation. From these results, it is possible that the C348T mutation and the A504C mutation respectively reside on independent alleles.

As shown above, the present inventors have analyzed mutations of the gene encoding the CD38 protein involving insulin secretion at insulin-producing cells. Then, after obtaining the results, the present inventors have further carried out comparative studies between the diabetic and non-diabetic groups for incidence rates of genetic mutations of the gene. The obtained results show that in the case of the mutations of exon 3 Arg140Trp, exon 7 Ser264Leu, and intron 7 −28G/A, all of which at least will probably cause loss of expression and function of the CD38 protein, the total incidence rate of those mutations was 6.1% (46/757 cases), demonstrating that the rate was significantly high as compared with 2.4% of the non-diabetic group (5/205 cases). It has been reported that the total mutation incidence rate of the mitochondrial gene in the diabetic group was about 2%, whose mutations are regarded as diabetic causing mutations within a single gene. In contrast, the mutation incidence rate of the CD38 gene encoding the CD38 protein was 6.1% for the diabetic group, indicating that the mutation incidence rate of the CD38 gene was extremely high in the diabetic group as compared with that of the mitochondrial gene. Further, when this was compared with other incidence rates reported so far, the incidence rate of 6.1% was likewise outstandingly high. It is well known that particular mutations could accumulate or be distributed unevenly from country to country or from region to region. Carrying out such studies and analysis of the diabetic subjects on a large scale may lead to finding much higher incidence rates of those 3-type genetic mutations or to finding of some other mutations of the CD38 gene other than the 3-type genetic mutations. Furthermore, use of the sum of the total abnormalities of the CD38 gene could identify a much higher number of diabetic patients than separate use of each mutation. Thus, the above 3-type genetic mutations or possible new mutations should not be used separately, but should be handled as mutations of the CD38 gene as the whole, which inevitably would greatly enhance usefulness of those markers for detecting risk factors for diabetic onset.

[Effects of the Invention]

Accorindg to the present invention, the means for detecting risk factors for diabetic onset using a gene is provided.

What is claimed is:

1. A method of detecting a risk factor for diabetic onset in an individual through detection of genetic abnormality of gene CD38.

2. A method of detecting a risk factor for diabetic onset according to claim 1, wherein sites of abnormality of the gene CD38 include the site encoding arginine at amino acid residue 140 of CD38 protein encoded by the CD38 gene; the site encoding serine at amino acid residue 264; and guanine at nucleotide position −28 in intron 7.
